(19) United States
(12) Patent Application Publication
(10) Pub. No.: US 2010/0151508 A1
(43) Pub. Date: Jun. 17, 2010

(54) COMPOUNDS

(75) Inventors: Paul Kong Thoo Lin, Aberdeenshire (GB); Charles Stuart Bestwick, Aberdeenshire (GB)

Correspondence Address: TAFT, STETTINIUS & HOLLISTER LLP, SUITE 1800, 425 WALNUT STREET, CINCINNATI, OH, 45202-3957, US

(73) Assignee: THE ROBERT GORDON UNIVERSITY, ABERDEEN, GB (unknown)

(21) Appl. No.: 12/515,285

(22) PCT Filed: Nov. 21, 2007
(86) PCT No.: PCT/GB07/04451

§ 371 Date: Jan. 26, 2010

(30) Foreign Application Priority Data
Nov. 21, 2006 (GB) 0623174.0

(51) Int. Cl. C12Q 1/02; C07D 311/22

(52) U.S. Cl. 435/29; 549/400

(57) ABSTRACT

Flavonoid-type compounds of the Formula (II); in which R¹ is alkyl C₂ to C₂₀, R², R³, R⁴ and R⁵ are any one or more of hydrogen, alkyl, alkenyl, alkynyl, phenyl or benzyl, especially 7-decyl-3-hydroxy-2-(3,4,5-trihydroxyphenyl)-4H-1-benzopy-ran-4-one or 7-ethyl-3-hydroxy-2-(3,4,5-trihydroxyphenyl)-4H-1-benzopyran-4-one provide fluorescent probes and antioxidants, and are useful in discriminating healthy and stressed cells.

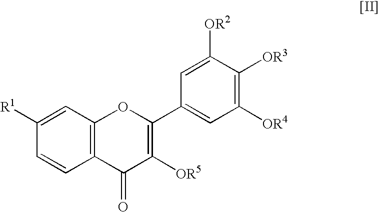

This invention relates to analytical reagents, and provides new uses for flavonoid compounds, especially as fluorescent probes.

### BACKGROUND TO THE INVENTION

Fluorescent organic molecules have widespread analytical potential for the real time measurement of metabolic processes. Fluorescence may be measured by conventional fluorimetry, flow cytometry and fluorescence microscopy, with the latter offering reaction localisation, as well as quantification. Flexibility of analysis through either ‘state of the art’ or more routinely available laboratory equipment, high sensitivity and, when compared with radiolabelling, fewer health and safety concerns, has ensured that fluorescent probes are a well established and rapidly expanding area of commercial product development [e.g. Invitrogen-Molecular Probes].

An important area of fluorescent probe development is that of oxidative stress research. Oxidative stress is implicated as a critical feature of many major chronic and acute health conditions and is a key analytical target within the biomedical and wider bio-science community. Unfortunately, many currently available probes suffer from either a lack of direct interaction with orchestrators of oxidative stress or have complicated localisation characteristics that may confound their analysis.

One such known fluorescent organic molecule is myricetin. Myricetin is a naturally occurring flavonoid that is found in, for example, grapes, berries, fruit, nuts, vegetables, herbs and red wine. Myricetin [A] is 3,5,7-trihydroxy-2-(3,4,5-trihydroxyphenyl)-4H-1-benzopyran-4-one:

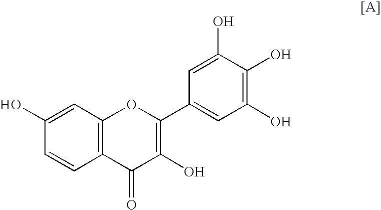

### SUMMARY OF THE INVENTION

Whereas, International Patent Application No. WO 2004/007475 describes novel flavonoid compounds and their use as therapeutic antioxidants, it has now surprisingly been found that, in addition to the properties described in the prior art, the compounds disclosed in WO 2004/007475 also possess, inter alia, improved fluorescent properties over myricetin.

Thus, according to the invention there is provided the use a compound of formula I:

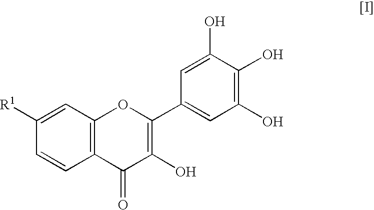

in which R¹ is C₂ to C₂₀ alkyl, as a fluorescent probe.

In a preferred embodiment of the present invention R¹ is a straight chain saturated alkyl group. Preferably, R¹ is C₂ to C₁₀ alkyl. In an especially preferred embodiment of the invention, the compound of formula I is one in which R¹ is either C₂ alkyl or C₁₀ alkyl.

Therefore, in an especially preferred aspect of the invention there is provided the use of 7-decyl-3-hydroxy-2-(3,4,5-trihydroxyphenyl)-4H-1-benzopyran-4-one (KB861) as a fluorescent probe.

In an alternative especially preferred aspect of the invention there is provided the use of 7-ethyl-3-hydroxy-2-(3,4,5-trihydroxyphenyl)-4H-1-benzopyran-4-one (KB474) as a fluorescent probe.

According to a further aspect of the invention there is particularly provided the use of a compound of formula I as a fluorescent probe for the detection of oxidative stress. Such use may be employed in determining or mitigating cellular damage or dysfunctionality due to exposure of cells to an oxidising agent.

Preferably, the probe is of the formula I wherein R¹ is one of the C₂ to C₁₀ alkyl groups.

Therefore, in an especially preferred aspect of the invention there is provided the use of 7-decyl-3-hydroxy-2-(3,4,5-trihydroxyphenyl)-4H-1-benzopyran-4-one (KB861) as a fluorescent probe for the detection of oxidative stress.

In an alternative especially preferred aspect of the invention there is provided the use of 7-ethyl-3-hydroxy-2-(3,4,5-trihydroxyphenyl)-4H-1-benzopyran-4-one (KB474) as a fluorescent probe for the detection of oxidative stress.

The compounds of formula I are either known per se or may be manufactured using the processes as described in WO 2004/007475 which is incorporated herein by reference.

The compounds of the invention are useful, inter alia, as probes and/or fluorescent markers, e.g. for in vitro use in assessing cell condition and functional status.

In a yet further aspect of the invention there is provided a method of measuring the oxidative stress of a cell which comprises the use of a compound of formula I as hereinbefore described. Said use may involve introducing a compound of formula I to a viable target cell, and examining the cell under conditions conducive to fluorescence, and determining whether fluorescence occurs. The determination may be by way of a comparison with cells whose oxidative stress condition has been predetermined. The determination may be a measurement of intensity of fluorescence.

In a preferred method of measuring oxidative stress, a reagent having the formula I wherein R¹ is one of the C₂ to C₁₀ alkyl groups is introduced to the cell. In an especially preferred embodiment of the invention, the reagent compound of formula I is one in which R¹ is either C₂ alkyl or C₁₀ alkyl.

Therefore, in an especially preferred aspect of the invention there is provided the use of 7-decyl-3-hydroxy-2-(3,4,5-trihydroxyphenyl)-4H-1-benzopyran-4-one (KB861), or 7-ethyl-3-hydroxy-2-(3,4,5-trihydroxyphenyl)-4H-1-benzopyran-4-one (KB474) as a reagent in a method of evaluating oxidative stress of a cell.

Thus the invention in a further aspect provides an in vitro test for discriminating healthy and unhealthy cells in vivo that comprises, providing at least one reagent of the formula I described hereinbefore, introducing said reagent to cells from a subject, examining the cells under conditions conducive to fluorescence, measuring the intensity of fluorescence, and correlating the measurement with a reference corresponding to cells of a healthy subject.

A kit for use in such an in vitro test, wherein, the reagent comprises a compound of the formula I wherein R¹ is selected from C₂ to C₁₀ alkyl groups, preferably a compound in which R¹ is either C₂ alkyl or C₁₀ alkyl.

More preferably the reagent comprises 7-decyl-3-hydroxy-2-(3,4,5-trihydroxyphenyl)-4H-1-benzopyran-4-one (herein identified as “KB861”) or 7-ethyl-3-hydroxy-2-(3,4,5-trihydroxyphenyl)-4H-1-benzopyran-4-one (herein identified as “KB474”).

The invention will now be described by way of example only and with reference to the accompanying drawings in which:

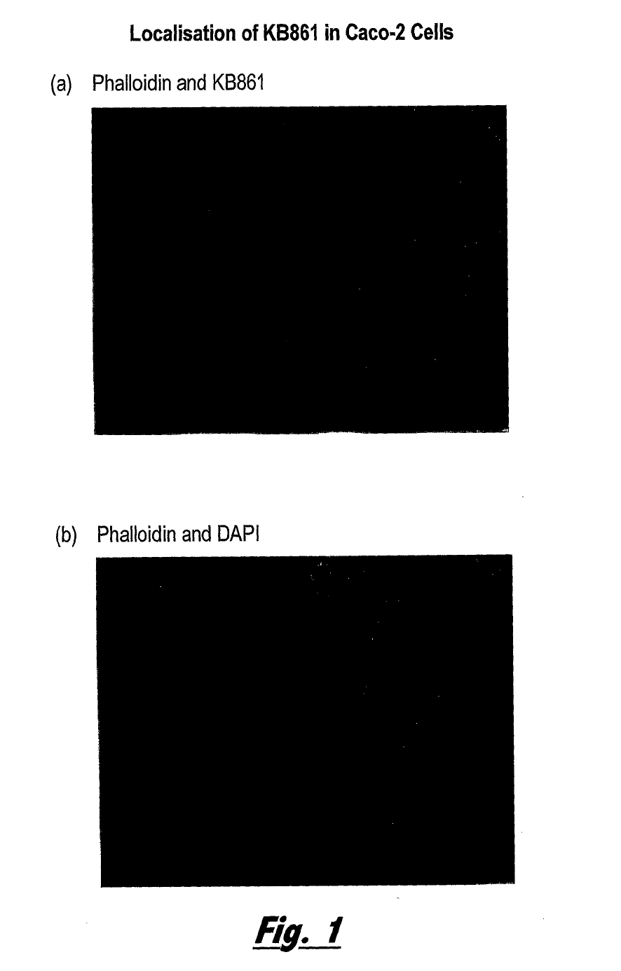

FIG. 1 shows the confocal images of the localisation of a compound “KB861” utilised in accordance with the invention in Caco-2 cells;

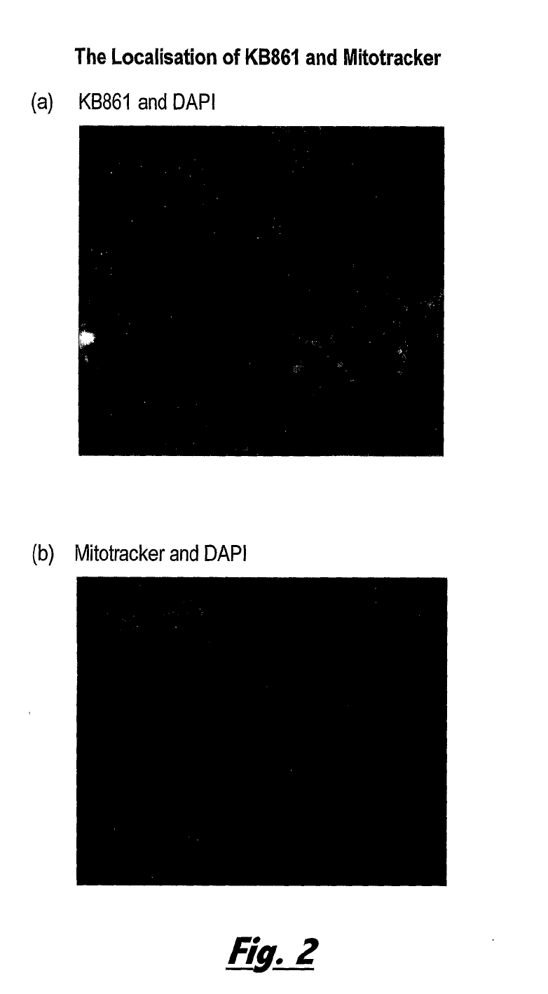

FIG. 2 shows the confocal images of the localisation of a compound “KB861” utilised in accordance with the invention and a known fluorescent reagent “Mitotracker”;

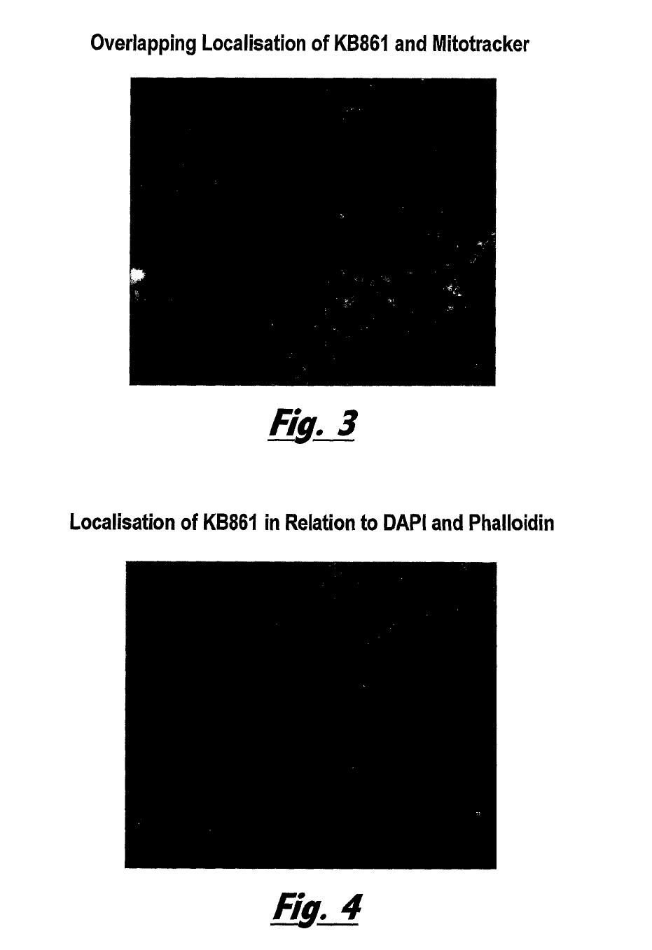

FIG. 3 shows an overlapping image of the localisation of a compound “KB861” utilised in accordance with the invention and “Mitotracker”;

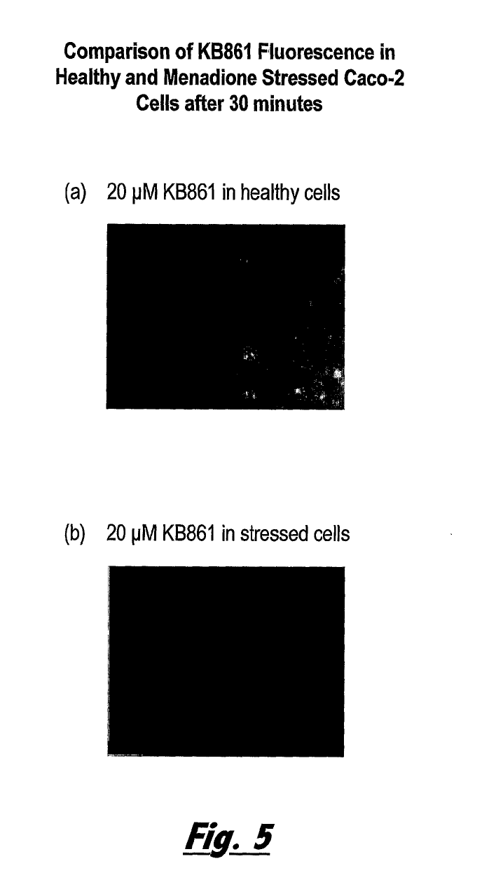

FIG. 4 shows the confocal images of the localisation of a compound “KB861” utilised in accordance with the invention in relation to known fluorescent reagents DAPI and Phalloidin;

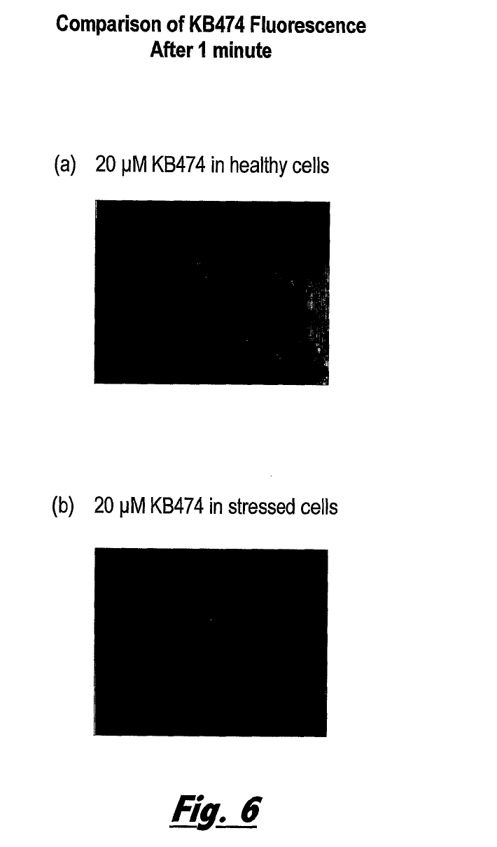

FIG. 5 shows a comparison of “KB861” fluorescence in healthy and menadione stressed Caco-2 cells after 30 minutes;

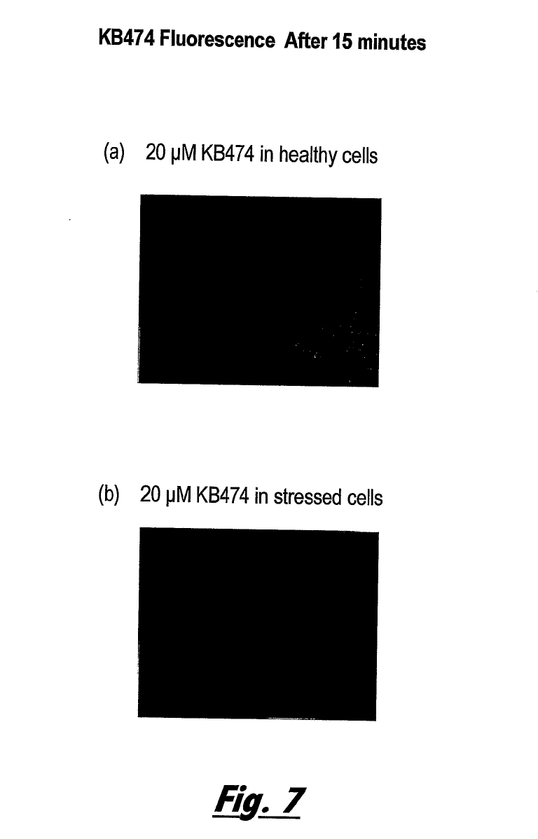

FIG. 6 shows a comparison of fluorescence of a compound “KB474” utilised in accordance with the invention in healthy and menadione stressed Caco-2 cells after 1 minute;

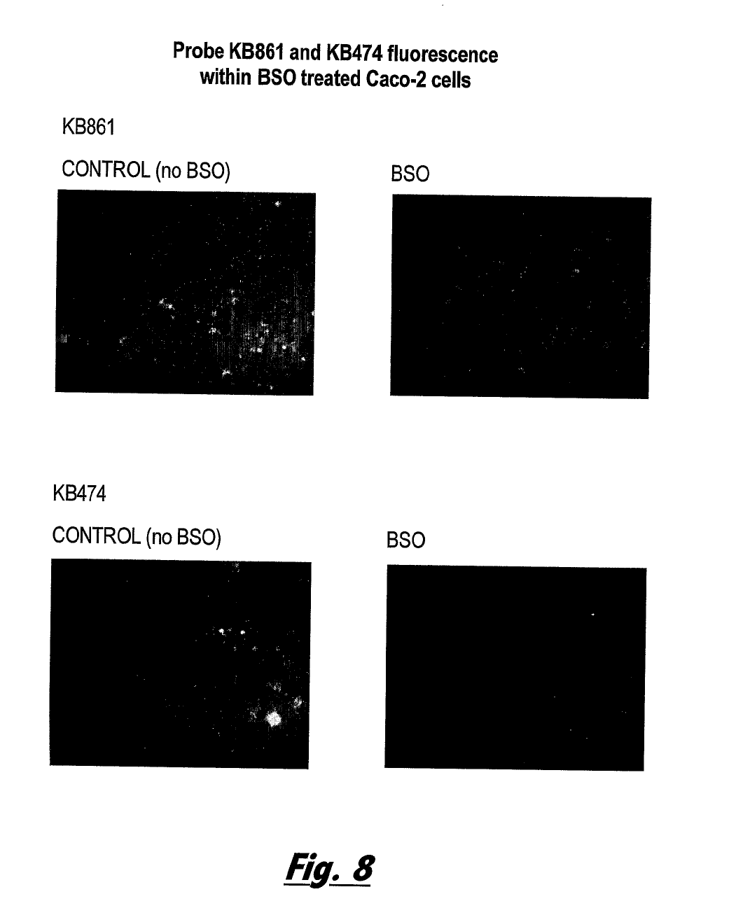

FIG. 7 shows a comparison of “KB474” fluorescence in healthy and menadione stressed Caco-2 cells after 15 minutes;

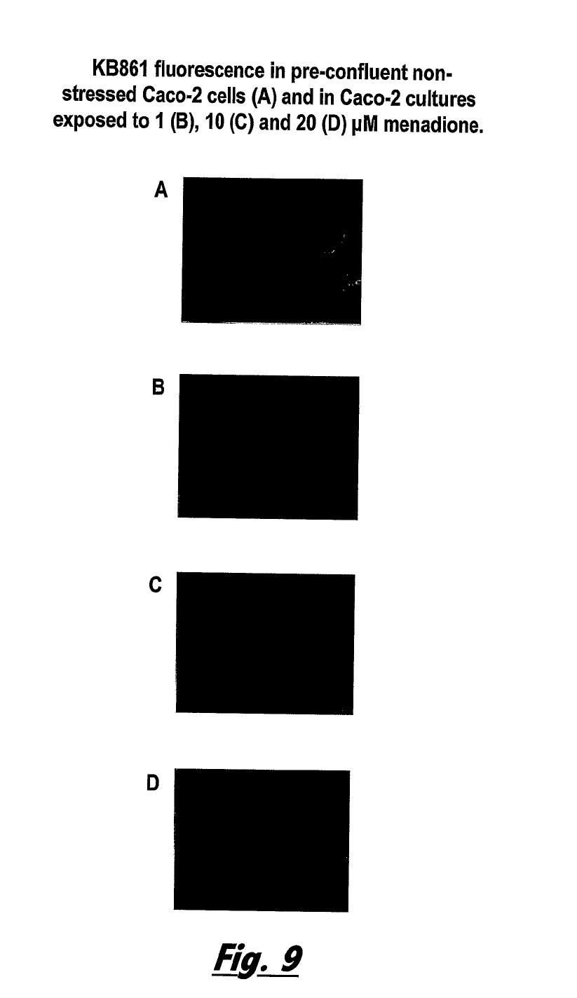

FIG. 8 is an illustration of the fluorescence within BSO treated Caco-2 cells with “KB861” and “KB474” probes;

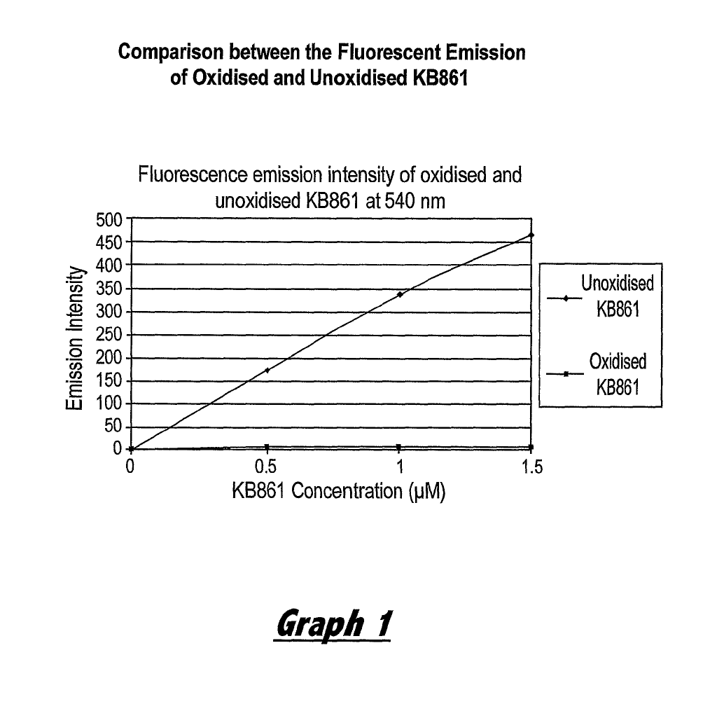

FIG. 9 shows a comparison of KB861 fluorescence in pre-confluent non-stressed Caco-2 cells (A) and in Caco-2 cultures exposed to 1, 10 and 20 μM menadione;

Graph 1 is a comparison between the fluorescent emission of oxidised and unoxidised KB861.

### SYNTHESIS EXAMPLES

### Synthesis Example 1

Synthesis of KB-861

Step 1

### 2-hydroxy-4-iodoacetophenone

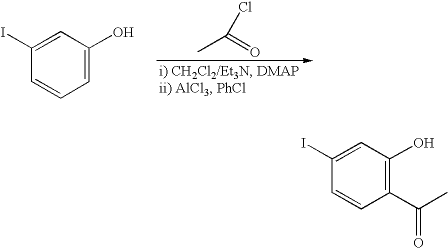 [0036] i) To a stirring solution 3-iodophenol (26.06 g, 118 mmol) and triethylamine (18 mL, 129 mmol) and DMAP (N,N-dimethyl-4-amino-pyridine, 0.62 g, 5 mmol) in dichloromethane (250 mL) under nitrogen at 0° C. was added dropwise, acetyl chloride (9 mL, 127 mmol). The reaction was allowed to stir at RT for 2 hr, washed with sat. sodium bicarbonate solution (200 mL). The organic layer was dried over magnesium sulphate, filtered and the solvent removed under vacuo to give 3-iodo-phenyl acetate as pale yellow solid (30.20 g, 90%).
ii) Aluminium chloride (31 g, 232 mmol) was added to a solution of 3-iodo-phenyl acetate (14.82 g, 90 mmol) in chlorobenzene (250 mL) under nitrogen. The mixture was heated to 140° C. for 90 hr then cooled. Reaction mixture was poured into ice/water and then filtered. The residue was washed with dicholoromethane. The filtrate the extracted with twice with dichloromethane. The combined organic layers were washed with 10% KOH (3×100 mL). The combined aqueous layers were acidified with 6N HCl and then extarcted with dichloromethane (3×75 mL). All the organic layers were dried with Magnesium Sulphate. Removal of the solvent gave product as a brown solid (22.3 g, 69%).

Step 2

### Synthesis of 4-Benzyloxy-3,5-dimethoxy-benzaldehyde

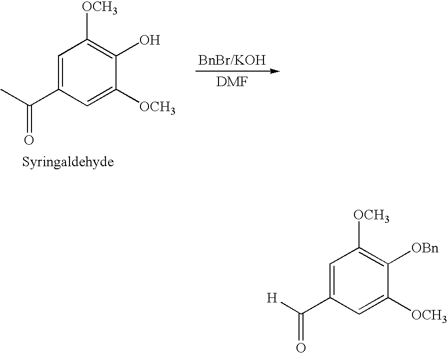

To a stirring suspension of syringaldehyde (25.19 g, 138 mmol) and K₂CO₃ (38.14 g, 276 mmol) in DMF (500 mL) was added benzyl bromide (20 mL, 168 mmol). The reaction was stirred for 25 h, and then poured in dichloromethane. The organic layer was washed with water (5×) and then dried. Removal of the solventgave a pink oil which was recrystallised from hexane (32.9 g, 87%).

Step 3

### 2′Hydroxy4′iodo4benzyloxy3,5dimethoxychalcone

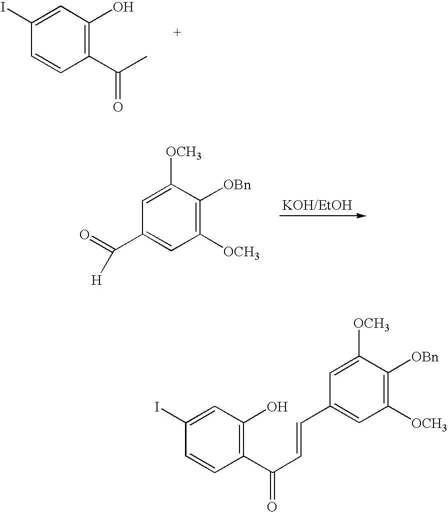

A solution of ketone (0.73 g, 2.8 mmol), 4-Benzyloxy-3,5-dimethoxy-benzaldehyde(0.911, 3.3 mmol) and Potassium hydroxide (0.42 g, 7.5 mmol) in ethanol (10 mL) were stirred for 46 hr to give a brown oil. The reaction was acidified with HCl (2M) and extracted with ethyl acetate (2×). The organic layers were washed with 10% sodium bisulphite solution (3×) and sat. sodium bicarbonate solution, dried with magnesium sulphate. Evaporation of the solvent afforded a solid. The latter was recrystallised with methanol to give chalcone as yellow crystals (1.06 g, 74%).

Step 4

### 3-Benzyloxy-7-iodo-2-(4-benzyloxy-3,5-dimethoxyphenyl)-Chromen-4-one

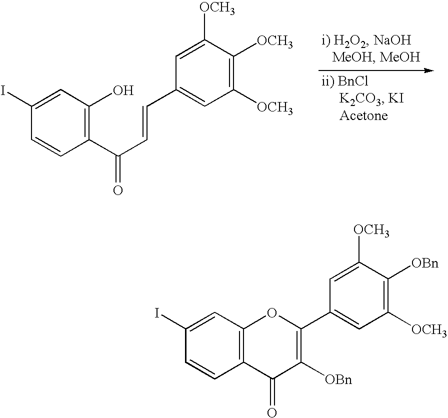 [0046] i) A solution of the above chalcone (0.85 g, 1.6 mmol), sodium hydroxide (2.2 mL, of 16% solution in water, 8.8 mmol) and hydrogen peroxide (2.2 mL of a 15% in water, 9.7 mmol) in methanol (17 mL). The solution was stirred at 0° C. and then sealed in the refrigerator for 26 hr. The reaction was acidified with HCl (2M) and extracted with dichloromethane (2×). The organic layers were dried and after evaporation of the solvent, afforded a yellow solid (0.84 g, 96%).
ii) To a stirring suspension of the above product, 3-hydroxy-7-iodo-(4-benzyloxy-3,5-dimethoxyphenyl)-chromen-4-one (5 g, 9 mmol), K₂CO₃ (6.2 g, 45 mmol), KI (0.64 g, 4 mmol) and Benzylchloride (1.7 mL, 15 mmol) in acetone (150 mL) under nitrogen was refluxed for 19 hr. The reaction was filtered and the filtrate was reduced under vacuo. The solis was recrystallised from isopropoanol to give product as a white solid (5.77 g, 99%).

Step 5

### 3-Benzyloxy-2-(4-benzyloxy-3,5-dimethoxy-phenyl)-7-decyl-chromen-4-one

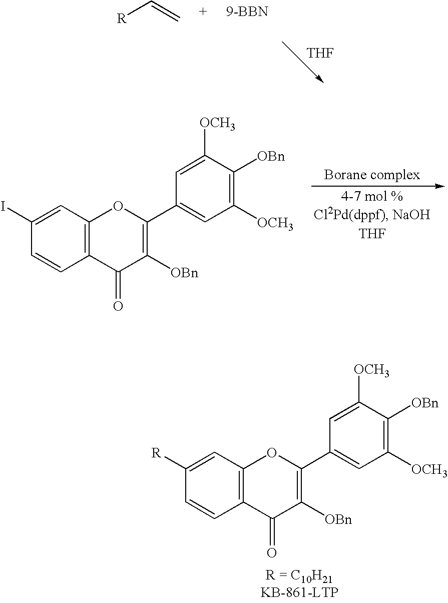

A solution of 1-decene (0.176 g, 1.3 mmol) and 9-BBN (1.35 mmol) in THF (4.7 mL) was stirred for 6 hr, then the iodocompound (0.56 g, 0.9 mmol) in THF (5 mL), NaOH (1.1 mL, 3M solution) and dichioropalladium (dppf, 27 mg) were added. The solution was left refluxing for 15 hr. Work up and column chromatography gave product 0.339 g, 59%).

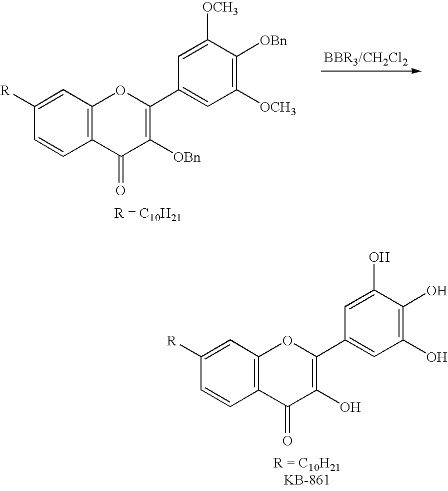

The above compound (0.504 g, 1.4 mmol) was added to boron tribromide (10 mmol) in dichloromethane (60 mL) dropwise at 0° C. The mixture was warmed to room temperature and then stirred for 22 hr. Solution was cooled to and methanol added (50 mL). The reaction was refluxed for 2 hr. All solvent was removed under vacuo to give a black solid. Water can be added followed by sonication and left overnight. Solid was collected as the product.

### Synthesis Example 2

Synthesis of KB-474

Step 1

### 4′-Ethyl-2′-hydroxy-acetophenone

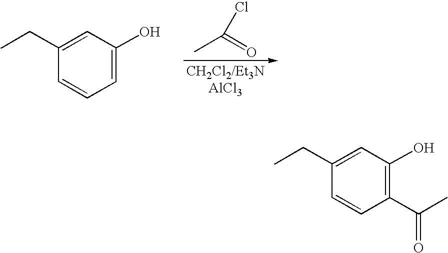

To a stirring solution 3-ethyl Phenol (9.95 g, 81 mmol) and triethylamine (14 mL, 100 mmol) in dichloromethane (100 mL) under nitrogen at 0° C. was added dropwise, acetyl chloride (7 mL, 98 mmol). The reaction was allowed to stir at RT for 3 hr, washed with sat. sodium bicarbonate solution (100 mL). The organic layer was dried over magnesium sulphate, filtered and the solvent removed under vacuo to give 3-ethyl-phenyl acetate as pale yellow solid (13.3 g, 97%).

To aluminium chloride (23 g, 172 mmol) was added 3-ethyl-phenyl acetate (14.82 g, 90 mmol) dropwise. The mixture was heated to 130° C. for 150 min then cooled. HCl (2M, 50 mL) was added slowly and mixture stirred for 45 min and then poured into HCl (2M, 85 mL). The resulting solution was extracted with diethylether (2×). the combined organic layers was washed with water, 1% sodium carbonate, water followed by drying with Magnesium Sulphate. Removal of the solvent gave product as a brown oil (10.8 g, 97%).

Step 2

### 1-(4-Ethyl-2-hydroxy-phenyl)-3-(3,4,5-trimethoxyphenyl)-propenone

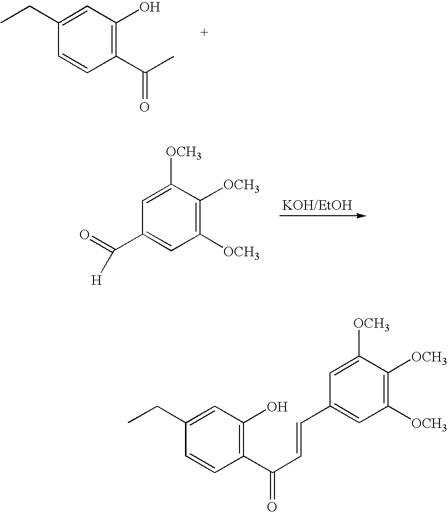

A solution of ketone (5.00 g, 30 mmol), 3,4,5-trimethoxy benzaldehyde (7.20 g, 37 mmol) and Potassium hydroxide (4.21 g, 7.5 mmol) in ethanol (145 mL) were stirred for 200 hr. The reaction was acidified with HCl (2M) and extracted with ethyl acetate (2×). The organic layers were washed with 10% sodium bisulphite solution (3×) and sat. sodium bicarbonate solution, dried with magnesium sulphate. Evaporation of the solvent afforded a solid. The latter was boiled with ethanol and the resulting solid collected after filtration to give a brown tar (9.62 g, 92%) and was used in the next step without further purification.

Step 3

### 7-Ethyl-3-hydroxy-2-(3,4,5-trimethoxy-phenyl)-Chromen-4-one

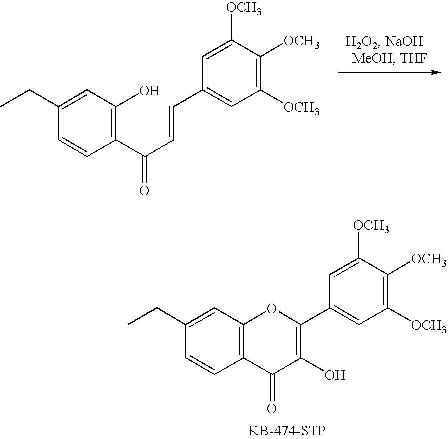

A solution of the above product (1.6 g, 4.7 mmol), sodium hydroxide (6.5 mL, of 16% solution in water, 26 mmol) and hydrogen peroxide (6.5 mL of a 15% in water, 29 mmol) in methanol (45 mL). The solution was stirred at 0° C. and then sealed in the refrigerator for 26 hr. The reaction was acidified with HCl (2M) and extracted with dichloromethane (2×). The organic layers were dried and after evaporation of the solvent, afforded a yellow solid (0.77 g, 47%).

Step 4

### 7-Ethyl-3-hydroxy-2-(3,4,5-trihydroxy-phenyl)-chromen-4-one

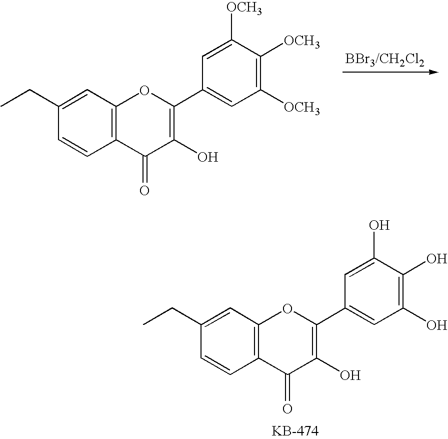

The above compound (0.504 g, 1.4 mmol) was added to boron tribromide (10 mmol) in dichloromethane (60 mL) dropwise at 0° C. The mixture was warmed to room temperature and then stirred for 22 hr. Solution was cooled to and methanol added (50 mL). The reaction was refluxed for 2 hr. All solvent was removed under vacuo to give a black solid. Water can be added followed by sonication and left overnight. Solid was collected as the product.

### EXPERIMENTAL EXAMPLES

### Experimental Example 1

### Fluorescence Emission of 3-hydroxy-2-(3,4,5-trihydroxyphenyl)-4H-1-benzopyran-4-one-yl Compounds

All the KBX compounds and Myricetin were prepared for analysis of their fluorescent properties (Table 1) using ethanol as the solvent, with the exception of KB861 which used tetrahydrofuran (THF). All measurements were taken at a compound concentration of 2.5 μM.

TABLE 1
Fluorescence Emission of Myricetin Derivatives

| | | Emission | | |
|---|---|---|---|---|
| Compound | Structure | λmax (nm) | intensity | |
| AOX861 | 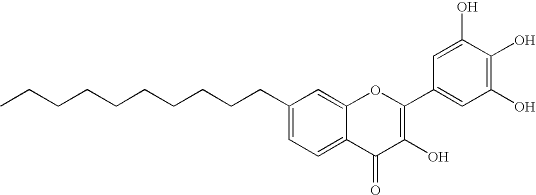 | 544.7 | 664.669 | 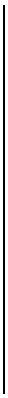 |
| AOX474 | 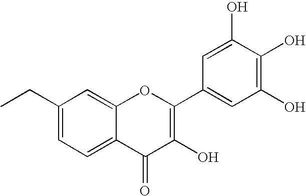 | 504.9 | 321.113 | 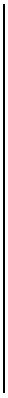 |
| Myricetin | 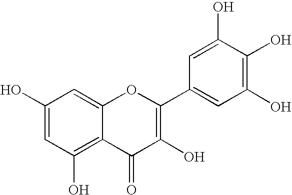 | 499.5 | 17.419 | 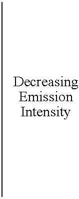 |
| AOX166 | 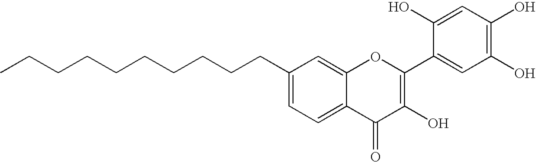 | 514.1 | 0.286 | 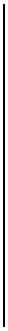 |
| AOX563 | 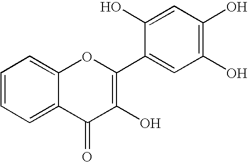 | 517.0 | 0.199 | 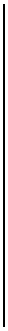 |
| AOX558 | 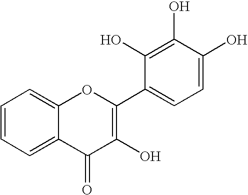 | 457.1 | 0.166 | 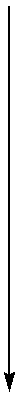 |

### 1.1. Localisation of 7-decyl-3-hydroxy-2-(3,4,5-trihdroxyphenyl)-4H-1-benzopyran-4-one (KB861)

Confocal microscopy was used to obtain detailed images of co-labelled Caco-2 human colon cancer cell lines in order to clarify the localisation of KB861.

Initially, confluent Caco-2 cells were co-labelled with KB861, DAPI and Phalloidin (FIG. 1). DAPI is commonly used for cell nucleus staining and emits a blue fluorescence. Phalloidin is a cell membrane dye that emits a red fluorescence, whereas KB861 produces a green fluorescence when excited.

Whereas it was anticipated that KB861 would target the cell membranes, initial confocal images showed no green fluorescence, which would be indicative of KB861, in the cell membrane. Instead, particulate green fluorescence could be observed only in the cytoplasmic region of the Caco-2 cells. The particular nature of the fluorescence suggested that KB861 was possibly localising in the mitochondria of the cells.

Therefore an experiment was designed using KB861, DAPI and Mitotracker to clarify the localisation of KB861 (FIG. 3). Mitotracker localises to the mitochondria of cells while emitting a red fluorescence when excited.

From the confocal images of FIG. 2 it was observed that both KB861 and Mitotracker gave very similar fluorescent staining of the Caco-2 cells. Both KB861 and Mitotracker had a particulate nature to their fluorescence and both dyes seemed to be localising in a very similar position in the Caco-2 cells (FIG. 3).

From these confocal microscopy experiments it was concluded that KB861 did not localise in the cell membrane as it was expected nor did it localise in the cell nucleus but it very likely concentrated in the mitochondria of the Caco-2 cells (FIG. 4).

### Experimental Example 2

Response of KB Components to Oxidative Stress

Due to the intense fluorescent properties of KB861, the possibility of using it as a probe to monitor cellular oxidative stress arose. Therefore a series of experiments were designed to test the reactivity of KB861 in vivo and in vitro to oxidative stress.

2.1 In Vitro Testing

A series of K5861 solutions (0.5, 1.0, and 1.5 μM) were prepared using THF. These solutions were then oxidised by excess Silver (II) Oxide (AgO) and analysed by fluorimetry. Another series of KB861 solutions of the same concentrations were prepared and analysed by fluorimetry in their natural unoxidised state (Graph 1).

It can be seen from Graph 1 that KB861 in its natural unoxidised state is highly fluorescent at 540 nm at low concentrations and that there is a linear relationship between the intensity of the fluorescent emission and the concentration of the sample. However, when KB861 is oxidised using AgO the fluorescence emission at 540 nm almost completely disappears. This indicates that, in vitro KB861 fluorescence at 540 nm is sensitive to exposure to a common cellular reactive oxygen species.

2.2 In Vivo Testing

A set of experiments was designed to investigate KB861 and its response to oxidative stress in vivo.

As KB861 fluorescence was diminished by hydrogen peroxide, the structurally similar and fluorescent compound 7-ethyl-3-hydroxy-2-(3,4,5-trihyroxyphenyl)-4H-1-benzopyran-4-one (KB474) was also tested for its reactivity to in vivo oxidative stress.

Initially, confluent Caco-2 cells were placed under stress using menadione then incubated with 20 μM KB861 for 30 minutes. The fluorescence of KB861 in stressed cells was then compared to that of KB861 in healthy cells using fluorescence microscopy (FIG. 5).

From this initial experiment it was observed that when KB861 was placed in stressed cells a decrease of 30-40% in fluorescence was noticed when compared to the fluorescence of the compound in healthy Caco-2 cells. The experiment was then repeated using KB474 to investigate whether these results could be replicated using a different but structurally similar compound.

Confluent Caco-2 cells were placed under stress using Menadione then incubated with 20 μM KB474 for 1 minute (FIG. 6) and 15 minutes (FIG. 7). The fluorescence of the compound in the stressed cells was then compared to its fluorescence in healthy Caco-2 cells.

It could be established from this experiment that placing KB474 into stressed cells had a striking effect on the intensity of the fluorescence emission. A decrease of at least 75% in KB474 fluorescence was apparent in the stressed Caco-2 cells. This decrease in fluorescence was greater than that observed for KB861. When comparing compound incubation conditions achieving a similar fluorescence distribution within non-stressed Caco-2 cultures, it is indicated that KB474 is more sensitive to the effects of cellular stress than KB861.

Pre-confluent, proliferating Caco-2 cultures were also exposed to menadione prior to staining with KB861 or KB474. Again, relative to control non-stressed cultures, KB861 or KB474 fluorescence was dramatically decreased in cultures exposed to menadione. KB474 fluorescence exhibited greater sensitivity to the oxidatively stressed environment.

The effect of menadione concentration on the intracellular fluorescence of compound KB861 and KB474 was also investigated. The extent of decrease in KB861 fluorescence correlated with the concentration of menadione exposure (FIG. 9) and thereby, putatively, the intensity of oxidative stress. A menadione dose dependent decrease in KB474 intracellular fluorescence was also observed.

2.3 Glutathione Depletion

To examine probe sensitivity to a less severe, more gradually provoked physiological oxidative stress within Caco-2 cells, cellular glutathione was depleted by progressive buthionine sulphoximine (BSO) exposure.

Increasing oxidative stress as a consequence of BSO exposure lead to a striking decrease in the fluorescence emission from cultures stained with KB861 or KB474. Again KB474 fluorescence exhibited greater sensitivity to oxidative stress.

1. The use of a compound of formula II; 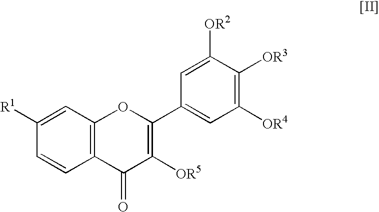
in which R¹ is alkyl C₂ to C₂₀, R², R³, R⁴ and R⁵ which may the same or different are selected from any one or more of hydrogen, alkyl, alkenyl, alkynyl, phenyl or benzyl as a fluorescent probe for the detection of oxidative stress.

2. The use of a compound of formula II; 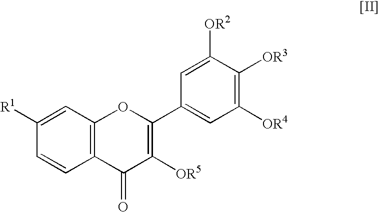
in which R¹ is alkyl C₂ to C₂₀, R² and R⁴ are methyl, R³ is benzyl, R⁵ is hydrogen as a fluorescent probe for the detection of oxidative stress.

3. The use according to any of claims 1 to 2, wherein R¹ is a straight chain saturated alkyl group.

4. The use according to any of claims 1 to 2, wherein R¹ is alkyl C₂ to C₁₀.

5. The use according to any of claims 1 to 2, wherein R¹ is either C₂ alkyl or C₁₀ alkyl.

6. The use of the compound 7-decyl-3-hydroxy-2-(3,4,5-trihydroxyphenyl)-4H-1-benzopyran-4-one as a fluorescent probe for the detection of oxidative stress.

7. The use of the compound 7-ethyl-3-hydroxy-2-(3,4,5-trihydroxyphenyl)-4H-1-benzopyran-4-one as a fluorescent probe for the detection of oxidative stress.

8. The use of a compound of formula II according to any of claims 1 to 7 for at least one of determining or mitigating cellular damage due to exposure of cells to an oxidising agent.

9. The use of a compound of formula II according to any of claims 1 to 7 as a fluorescent probe for the in vitro assessment of cell condition and functional status.

10. The use of a compound of formula II according to any of claims 1 to 7 as a fluorescent marker for the in vitro assessment of cell condition and functional status.

11. A method of measuring the oxidative stress on a cell which comprises the use of a compound of formula II; 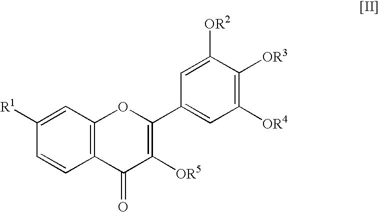
in which R¹ is alkyl C₂ to C₂₀, R², R³, R⁴ and R⁵ which may the same or different are selected from any one or more of hydrogen, alkyl, alkenyl, alkynyl, phenyl or benzyl.

12. A method of measuring the oxidative stress on a cell which comprises the use of a compound of formula II; 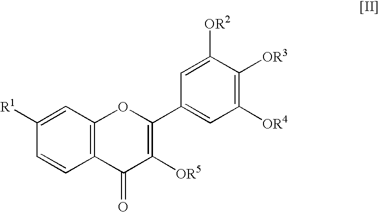
in which R¹ is alkyl C₂ to C₂₀, R² and R⁴ are methyl, R³ is benzyl, R⁵ is hydrogen.

13. A method according to any of claims 11 to 12, wherein R¹ is a straight chain saturated alkyl group.

14. A method according to any of claims 11 to 12, wherein R¹ is a C₂ to C₁₀ alkyl.

15. A method according to any of claims 11 to 12, wherein R¹ is either C₂ alkyl or C₁₀ alkyl.

16. A method of evaluating the oxidative stress on a cell using the compound 7-decyl-3-hydroxy-2-(3,4,5-trihydroxyphenyl)-4H-1-benzopyran-4-one as a reagent.

17. A method of evaluating the oxidative stress on a cell using the compound 7-ethyl-3-hydroxy-2-(3,4,5-trihydroxyphenyl)-4H-1-benzopyran-4-one as a reagent.

18. A method of measuring the oxidative stress of a cell which comprises the steps of
(i) introducing to a target cell a compound of formula II according to any of claims 11 to 17,
(ii) examining the cell under conditions conducive to fluorescence,
(iii) determining whether fluorescence occurs.

19. A method according to claim 18 wherein the determination is by way of a comparison with cells whose oxidative stress condition has been predetermined.

20. A method according to claim 18 wherein the determination is a measurement of intensity of fluorescence.

21. A method of providing an in vitro test for discriminating healthy and unhealthy cells in vivo that comprises the steps of
(i) providing at least one reagent of the formula II according to any of claims 11 to 17;
(ii) introducing said reagent to cells from a subject;
(iii) examining the cells under conditions conducive to fluorescence;
(iv) measuring the intensity of fluorescence;
(v) correlating the measurement with a reference corresponding to cells of a healthy subject.
